(19) United States
(12) Patent Application Publication
(10) Pub. No.: US 2016/0015373 A1
(43) Pub. Date: Jan. 21, 2016

(54) IMPREGNATED BRUSH APPLICATOR FOR MEDICAL ADHESIVES AND SEALANTS

(71) Applicant: Cyberbond LLC, Batavia, IL (US)

(72) Inventors: Joseph D. Russo, Palo Alto, CA (US); Joseph Silvestro, Geneva, IL (US); James East, St. Charles, IL (US); Mark Conway, Woodridge, IL (US)

(21) Appl. No.: 14/333,450

(22) Filed: Jul. 16, 2014

(51) Int. Cl. A61B 17/00; A61L 24/04

(52) U.S. Cl. CPC A61B 17/00491; A61L 24/046

(57) ABSTRACT

High viscosity medical adhesive formulations are provided with an applicator device that allows the medical adhesives to be easily sterilized, accurately dispensed and optimally cured for medical applications. The preferred medical adhesive formulations include 2-octyl cyanoacrylate and n-butyl-2-cyanoacrylate monomers in formulation with various additives. The medical adhesive can be formulated to be bio-absorbable and/or bio-degradable. Suitable thickeners, stabilizers, plasticizing agents, preservatives, reinforcing agents, colorants and anti-bacterial compounds may advantageously be included in the formulation. The formulation is preferably placed within a sealed aluminum or plastic tube. An applicator tip with plastic brush bristles is used for application and curing of the formulation. On one end, the applicator tip preferably has a cutting or piercing mechanism which is capable of breaking the seal on the tube. On the other end, the applicator tip brush bristles are coated and/or impregnated with a dry, solvent-free cyanoacrylate polymerization initiator and/or accelerator.

### FIELD OF THE INVENTION

This invention relates to precision applicator devices for high viscosity medical adhesives. More particularly, brush applicators are used to apply high viscosity cyanoacrylate formulations where the brush bristles are coated and/or impregnated with a dry compound to initiate and/or accelerate curing of the cyanoacrylate compositions.

### BACKGROUND OF THE INVENTION

Cyanoacrylate is the generic name for a family of strong, fast-acting adhesives with industrial, medical and household uses. Cyanoacrylates include methyl 2-cyanoacrylate, ethyl-2-cyanoacrylate (commonly sold under trade names such as “Super Glue” and “Krazy Glue”), n-butyl cyanoacrylate and 2-octyl cyanoacrylate (used in medical, veterinary and first aid applications).

Methyl and ethyl cyanoacrylates typically have a low viscosity and tend to be runny. They also cure quickly when exposed to the type of moisture often found on a substrate. This combination of runniness and quick curing make methyl and ethyl cyanoacrylates difficult to control. For this reason, there has been much effort in the cyanoacrylate art to make systems that allow methyl and ethyl cyanoacrylates to be applied in a controlled manner. One such system is described in Russo's U.S. Pat. Nos. 4,687,827 and 4,724,177 which was especially developed with artificial fingernail applications in mind. In the Russo system, a brush is pre-wetted with a solvent that slows the polymerization of methyl and ethyl cyanoacrylates. The pre-wetted brush is then used to apply these cyanoacrylates to a surface, such as a natural fingernail, without causing the cyanoacrylates to cure until the solvent evaporates. When instant bonding is desired, a curing accelerator is then sprayed onto the brushed cyanoacrylate. In the case of bonding artificial fingernails to natural fingernails, the curing accelerator is sprayed onto the cyanoacrylates after they have been brushed onto the natural fingernail but before the artificial fingernail is attached to the natural fingernail. Repensek's U.S. Pat. No. 4,844,102 discloses a system similar to those disclosed in the Russo patents except that, during the application process, the brush is dipped in a liquid mixture which includes both a solvent and a polymerization initiator. As in the Russo system, the solvent in Repensek's liquid mixture initially slows the polymerization of the cyanoacrylates but, when the solvent evaporates, Repensek's polymerization initiator is then able to accelerate the polymerization of the cyanoacrylates. While the Repensek patent tries to downplay the risks associated with his preferred ethyl acetate solvent, the types of liquid solvents used in the Russo and Repensek patents have known fire, irritant and/or toxicity risks.

Other cyanoacrylate applicator systems include Nentwick et al.'s U.S. Pat. Appl. Pub. No. 2008/0105580 which discloses an applicator tip for dispensing a cyanoacrylate-based adhesive from a reservoir. The applicator tip includes an opening offset and a distal end. The adhesive material is dispensed when pressure is applied to the applicator tip surface. Stenton et al's U.S. Pat. Appl. Pub. No. 2007/0147947 discloses an applicator for forming uniform thickness layers of liquid coating on a substrate surface. The applicator is characterized by controlling the dispensing of liquid through apertures incorporated within the applicator head. The applicator uses a supported thin layer of foam which assures uniform thickness of applied layers, which are substantially independent of the pressure applied to the applicator. Battisti et al's U.S. Pat. Appl. Pub. No. 2006/0282035 discloses a disposable swab applicator for containing and dispensing cyanoacrylate adhesive. The swab applicator is closed at one end and the cyanoacrylate composition is contained by a valve that can be easily opened when desired. The valve can be a ball, a bead or a capsule. The device can be heat sterilized using dry heat sterilization. Dewitt's U.S. Pat. No. 7,297,217 discloses a dispenser for application of a special low viscosity cyanoacrylate adhesive which is used for the manufacture and repair of wooden furniture. The dispenser is provided with a closure member having a metallic pin which penetrates into the discharge opening while the closure member is being secured thereon. Mainwaring et al.'s U.S. Pat. No. 6,779,657 discloses a single-use applicator assembly for applying and dispensing cyanoacrylate monomeric adhesive material. The applicator comprises a base with at least one sealed container and an applicator tip at least partially disposed in the container such that the tip of the applicator has access to the adhesive material. Quintero's U.S. Pat. No. 6,547,467 discloses a micro-applicator for dispensing and applying cyanoacrylate-based adhesive. The micro-applicator comprises a handle portion, a micro-reservoir at the applicator tip to hold about 20 microliter or less of adhesive material. The applicator tip may include a (non-curative) loop, a spatula, a rolling ball, a grate, a porous material, or a brush. Lier et al's U.S. Pat. No. 5,649,648 discloses a packaging system for free-flowing material such as cyanoacrylate adhesive. The package comprises a container and a closable applicator point fitted on its outlet aperture. The container is made of an extruded receptacle aluminum which springs back when the pressure is released. Schaefer et al.'s U.S. Pat. No. 4,685,591 discloses a packaging tube that is suitable for storing and dispensing products containing substantial fractions of cyanoacrylates. The tube sidewall is made of multi-layer sheet material and a covering strip is placed over the inside surface the tube. Stock's U.S. Pat. No. 4,413,753 discloses a self-draining tip for dispensing cyanoacrylate adhesives. The dispensing tip includes a single or segmented constant diameter passageway having sharp-edged annular terminations for dispensing. U.S. Pat. Appl. Pub. 2009/0050649 Al discloses a cyanoacrylate consumer product nozzle assembly comprising a brush insert for application of the dispensed product to a surface. The product is so highly stabilized to prevent curing that the brush can be capped until further use is required.

Since the original methyl (C₅H₅NO₂) and ethyl (C₆H₇NO₂) forms of cyanoacrylates irritated human tissue, it was not until N-butyl-2-cyanoacrylate was developed in the 1970's that cyanoacrylates began to be commonly used for medical applications. N-butyl-2-cyanoacrylate monomer compositions have been used in cartilage and bone grafting, coating of corneal ulcers in ophthalmology, repair of damaged ossicles in otolaryngology, coating of aphthous ulcers, embolization of gastrointestinal varicies and embolization in neurovascular surgery. 2-octyl cyanoacrylate monomer-based cyanoacrylate compositions came into use in the 1990's and were demonstrated to have less toxicity than N-butyl-2-cyanoacrylate.

Medical products such as liquid bandages, wound dressings, skin bonding surgical adhesives, sealants, bioactive agent release matrixes and implants require that dispensing of the cyanoacrylates be carefully controlled. U.S. Pat. Nos. 5,928,611 and 6,099,807 disclose dispensing devices developed by Closure Medical Corporation for their DERMABOND® cyanoacrylate products (now sold by Ethicon, a subsidiary of Johnson & Johnson Corporation). These patents disclose that the cyanoacrylate is stored in a sealed tube or frangible vial. A frangible vial is a tube typically made of glass. The cyanoacrylate can be applied by first breaking the seal or frangible vial and then squeezing out the cyanoacrylate. The cyanoacrylate then passes through a porous plug on the applicator tip with is coated with an accelerator/initiator for accelerating the curing of the cyanoacrylate. Other patents and patent applications obtained by Closure Medical Corporation pertaining to their DERMABOND® products include U.S. Pat. Appl. Pub. No. 2005/0175395 as well as U.S. Pat. Nos. 7,306,390 and 6,705,790 to Quintero et al. disclosing an applicator assembly for dispensing adhesive material. The applicator comprises first and second body portions, a frangible ampule container for adhesive, and a breaking member to rupture the container for dispensing the adhesive material. U.S. Pat. Nos. 6,960,040, 6,494,896, and 6,340,097 to D'Alessio et al. disclose package assembly suitable for laparoscopic or endoscopic surgery.

While the DERMABOND® medical adhesive dispenser is in widespread commercial use, it has a number of disadvantages. For example, when a glass frangible vial is broken, it produces shards of glass that can expose the user or patient to risk. As published by the FDA in the Maude Adverse Event Report, the glass vial of the DERMABOND® applicator was crushed and the shard protruded through the tube, penetrated the gloves and pierced the hands of the medical professionals or patients. The shard penetration adverse effect of the DERMABOND® product occurred repeatedly since it was marketed. U.S. Pub. Pat. Appl. No. 2013/0004230 assigned to OptMed, Inc. claims to overcome the DERMABOND® shard problem by placing a filter element in the dispenser to capture hazardous shards.

Another problem associated with the DERMABOND® dispenser is the flow restrictions caused by the porous applicator tip. While cyanoacrylates of moderate viscosity can flow through the DERMABOND® porous applicator tip, the tip is too tightly packed to allow adequate flow of high viscosity cyanoacrylates, such as thickened 2-octyl cyanoacrylate. Because highly stabilized (and thus slow to cure) cyanoacrylate formulations are more stable from exposure to heat or irradiation, such as electron beam, gamma and/or x-ray irradiation, the DERMABOND® porous applicator plug, that requires low viscosity, makes the cyanoacrylates transferred to skin more exothermic in curing. This is because co-elution of curative with thin cyanoacrylate poses the problem of the curative in the plug mixing with the recommended first, partial dose of cyanoacrylate having a highly exothermic curative component with a low adhesive mass. The result is serious skin and tissue burning complaints, which have been documented.

Expense is an additional drawback of the DERMABOND® system. The cost of placing cyanoacrylates in a frangible vial and inserting them into a dispenser with a porous applicator tip limits the ability of poorer nations to purchase DERMABOND® medical cyanoacrylate adhesives for widespread use.

### SUMMARY OF THE INVENTION

The present invention provides medical adhesive formulations and dispensers that allow the medical adhesives to be easily sterilized, accurately dispensed and optimally cured for medical applications. The preferred medical adhesives include 2-octyl cyanoacrylate and n-butyl-2-cyanoacrylate monomers in a formulation with various additives. To increase viscosity, polymeric thickeners such as polyvinyl acetate (PVAc) or poly 2-ethyl hexyl methylmethacrylate (PEHMA) are preferably added to the cyanoacrylate composition. In some formulations, the thickener constitutes 3% to 15% of the cyanoacrylate composition by volume. Suitable stabilizers, plasticizing agents, preservatives, reinforcing agents, colorants and anti-bacterial compounds may also advantageously be used in the medical adhesive formulation. Also, the medical adhesives can be formulated to be bio-absorbable and/or bio-degradable.

The high viscosity formulation of the present invention is preferably placed within a sealed aluminum or plastic tube. An applicator tip with plastic brush bristles is preferably used for application and rapid curing of the high viscosity cyanoacrylate formulations. On one end, the applicator tip preferably has a cutting or piercing mechanism which is capable of breaking the seal on the cyanoacrylate tube. In one embodiment, the applicator tip can be screwed onto the cyanoacrylate tube and will break the tube seal as it is screwed on.

On the other end, the applicator tip preferably has a brush with plastic bristles which are impregnated and/or coated with a dry, solvent-free cyanoacrylate polymerization initiator and/or accelerator. Suitable polymerization initiators and/or accelerators include, but are not limited to, calixarenes and oxacalixarenes, silacrowns, crown ethers, cyclodextrin and its derivatives, polyethers, aliphatic alcohol, various aliphatic carboxylic acid esters, benzoyl peroxide and amine compounds. During manufacture, the accelerators may be applied in liquid form to the brush bristles and then allowed to dry. The coated and/or impregnated initiators and/or accelerators of the present invention are used to enhance curing of the cyanoacrylate composition when it comes in contact with and flows through the coated and/or impregnated bristles.

Brush head bristles are used in the present invention both to allow the high viscosity cyanoacrylate composition to flow more easily and also to allow it to be accurately applied. In contrast to the DERMABOND® porous applicator tip, the brush bristles of the present invention provide little resistance to the flow of the cyanoacrylate formulations. Moreover, as the cyanoacrylate formulations of the present invention flow over and through the brush bristles, the brush bristles allow the cyanoacrylates formulations to be applied directly to the site of a wound with pinpoint precision. The combination of accurate application and well-timed curing allows greater control in the use of cyanoacrylates for medical applications.

### BRIEF DESCRIPTION OF THE DRAWINGS

FIG. 1 shows a brush applicator tip for the present invention.

FIG. 2 is a close-up, cutaway view of the FIG. 1 brush applicator tip.

FIG. 3 illustrates a kit which includes both the brush applicator tip and a metallic tube containing medical adhesives.

FIG. 4 provides a close-up, cut away illustration of how a threaded brush applicator tip of the present invention can be aligned with a metallic or plastic squeeze tube before being connected together.

FIG. 5 illustrates how the threaded brush applicator tip and metallic or plastic squeeze tube shown in FIG. 4 can be screwed together and how, in the process, the tube seal is cut away.

FIG. 6 illustrates an assembled medical adhesives dispenser of the present invention having a flat brush.

FIG. 7 illustrates an assembled medical adhesives dispenser of the present invention having an angled brush.

FIG. 8 is an exploded view of a plastic squeeze tube dispenser of the present invention prior to assembly.

FIG. 9 is a close-up, cut away view of the plastic squeeze tube dispenser of FIG. 8 after it has been assembled.

FIG. 10 is a perspective view of a bellows which can be used to help evenly dispense medical adhesives from a metallic squeeze tube.

FIG. 11 illustrates the medical adhesives dispenser of the present invention being used to seal a cut wound.

FIG. 12 illustrates the medical adhesives dispenser of the present invention being used to create a protective seal over an abrasion or ulcer wound.

### DETAILED DESCRIPTION OF THE PREFERRED EMBODIMENTS

The present invention includes both medical adhesive formulations and dispensers that are optimally configured to apply those medical adhesive formulations in a precise and cost-effective manner. The preferred medical adhesives are 2-octyl cyanoacrylate and n-butyl-2-cyanoacrylate monomers in a formulation with various additives. Nonetheless, other types of readily polymerizable (i.e., anionically polymerizable and/or free radical polymerizable) monomers may be used as adhesives in the present invention. For example, other types of 1,1-disubstituted ethylene monomers (e.g., a cyanoacrylate monomer) may be used. Cyanoacrylate monomers suitable for use in the present invention include, but are not limited to, 1,1-disubstituted ethylene monomers of the formula:

CHR═CXY (I)

wherein X and Y are each strong electron withdrawing groups, and R is H, —CH═CH₂ or, provided that X and Y′ are both cyano groups, or a C₁-C₄ alkyl group.

Examples of monomers within the scope of formula (I) include alpha-cyanoacrylates, vinylidene cyanides, C₁-C₄ alkyl homologues of vinylidene cyanides, dialkyl methylene malonates, acylacrylonitriles, vinyl sulfinates and vinyl sulfonates of the formula CH₂═CX′Y wherein X′ is —SO₂R′ or —SO₃R′ and Y′ is —CN, —COOR′, —COCH₃, —SO₂R or —SO₃R′, and R′ is H or hydrocarbyl.

Preferred monomers of formula (I) for use in this invention are alpha-cyanoacrylates. These monomers are known in the art and have the formula:

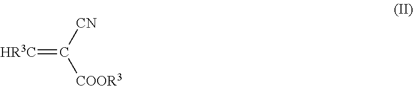

wherein R² is hydrogen and R³ is a hydrocarbyl or substituted hydrocarbyl group; a group having the formula —R⁴—O—R⁵—O—R⁶, wherein R⁴ is a 1,2-alkylene group having 2-4 carbon atoms, R⁵ is an alkylene group having 2-12 carbon atoms, and R⁶ is an alkyl group having 1-6 carbon atoms; or a group having the formula:

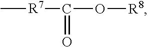

wherein R⁷ is

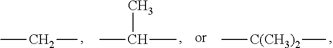

and R⁸ is an organic radical.

Examples of suitable hydrocarbyl and substituted hydrocarbyl groups include straight chain or branched chain alkyl groups having 1-16 carbon atoms; straight chain or branched chain C₁-C₁₆ alkyl groups substituted with an acyloxy group, a haloalkyl group, an alkoxy group, a halogen atom, a cyano group, or a haloalkyl group; straight chain or branched chain alkenyl groups having 2 to 16 carbon atoms; straight chain or branched chain alkynyl groups having 2 to 12 carbon atoms cycloalkyl groups; arylalkyl groups; alkylaryl groups; and aryl groups.

The organic moiety R⁸ may be substituted or unsubstituted and may be a straight chain, branched or cyclic, saturated, unsaturated or aromatic. Examples of such organic moieties include C₁-C₈ alkyl moieties, C₂-C₈ alkenyl moieties, C₂-C₈ alkynyl moieties, C₃-C₁₂ cycloaliphatic moieties, aryl moieties such as phenyl and substituted phenyl, and arylalkyl moieties such as benzyl, methylbenzyl and phenylethyl. Other organic moieties include substituted hydrocarbon moieties, such as halo (e.g., chloro-, fluoro- and bromo-substituted hydrocarbons) and oxy- (e.g., alkoxy substituted hydrocarbons) substituted hydrocarbon moieties. Preferred organic radicals are alkyl, alkenyl and alkynyl moieties having from 1 to about 8 carbon atoms, and halo-substituted derivatives thereof Particularly preferred are alkyl moieties of 4 to 8 carbon atoms. In the cyanoacrylate monomer of formula (II), R⁸ is preferably an alkyl group having 1-10 carbon atoms or a group having the formula -AOR⁹, wherein A is a divalent straight or branched chain alkylene or oxyalkylene moiety having 2-8 carbon atoms, and R⁹ is a straight or branched alkyl moiety having 1-8 carbon atoms. The preferred alpha-cyanoacrylate monomers used in this invention are 2-octyl cyanoacrylate, dodecyl cyanoacrylate, 2-ethylhexyl cyanoacrylate, N- butyl-2- cyanoacrylate, methyl cyanoacrylate, 3-methoxybutyl cyanoacrylate, 2-butoxyethyl cyanoacrylate, 2-isopropoxyethyl cyanoacrylate, or 1-methoxy-2-propyl cyanoacrylate, or a combination thereof.

The cyanoacrylate monomers used in the present invention can be prepared according to methods known in the art. Reference is made, for example, to U.S. Pat. Nos. 2,721,858 and 3,254,111, each of which is hereby incorporated by reference in its entirety. One such process includes, for example, reacting a cyanoacetate with formaldehyde in the presence of a basic condensation catalyst at an elevated temperature to produce a low molecular weight polymer. A de-polymerization (or cracking) step is followed under high temperature and high vacuum in the presence of acidic and anionic inhibitors, yielding a crude monomer that can be distilled under high temperature and high vacuum in the presence of radical and acidic inhibitors.

The medical adhesives of the present invention are preferably formulated with other compounds, such as thickeners, stabilizers, plasticizing agents, preservatives, reinforcing agents, colorants and anti-bacterial compounds. By using thickeners, the present invention provides freedom to formulate rheological benefits (e.g., high viscosity and thixotropy) into the adhesive formulation. Suitable thickeners include, but are not limited to, poly 2-ethyl hexyl methylmethacrylate (PEHMA), polycaprolactone, polyvinyl acetate (PVAc), copolymers of alkyl acrylate and vinyl acetate, polyalkyl methacrylates, polyalkyl acrylates, lactic-glycolic acid copolymers, lactic acid-caprolactone copolymers, polyorthoesters, copolymers of alkyl methacrylates and butadiene, polyoxalates, and triblock copolymers of polyoxypropylene flanked by two hydrophilic chains of polyoxyethylene. Thickening agents include a partial polymer of cyanoacrylate as disclosed in U.S. Patent Application Pub. No. 2009/0318583, and triblock copolymers of polyoxyalkylene as disclosed in U.S. Patent Application Pub. No. 2009/0317353. Preferably the thickening agent is miscible in cyanoacrylate monomer compositions at room temperature.

Suitable stabilizers may include, but are not limited to, perchloric acid, hydrochloric acid, hydrobromic acid, sulfur dioxide, toluenesulfonic acid, fluorosulfonic acid, phosphoric acid, ortho, meta, or para-phosphoric acid, trichloroacetic acid, and sulfuric acid. The stabilizer may be present in an amount of about 2 ppm to about 500 ppm, preferably about 10 ppm to about 200 ppm. Cyanoacrylate monomers may also be stabilized by using a combination of free radical and anionic stabilizers. Suitable free radical stabilizers include, without limitation, butylated hydroxy anisole (BHA), hydroquinone, catechol, hydroquinone monomethyl ether and hindered phenols such as butylated hydroxyanisol, 4-ethoxyphenol, butylated hydroxytoluene (BHT, 2,6-di-tert-butyl butylphenol), 4-methoxyphenol (MP), 3-methoxyphenol, 2-tert-butyl-4-methoxyphenol, and 2,2-methylene-bis-(4-methyl-6-tert-butylphenol).

The formulation may further include at least one plasticizing agent that imparts flexibility to the polymer formed from the monomer. The plasticizing agent preferably does not contain any moisture and should not adversely affect the stability of the adhesive compositions. Examples of suitable plasticizers include, but are not limited to, tributyl citrate (TBC), acetyl tributyl citrate (ATBC), dimethyl sebacate, diethylsebacate, triethyl phosphate, tri(2-ethyl-hexyl)phosphate, tri(p-cresyl) phosphate, diisodecyl adipate (DIDA), glyceryl triacetate, glyceryl tributyrate, dioctyl adipate (DICA), isopropyl myrisate, butyl sterate, lauric acid, trioctyl trimelliate, dioctyl glutatrate (DICG) and mixtures thereof. Tributyl citrate, diisodecyl adipate and acetyl tributyl citrate are preferred plasticizers which may be present in an amount of up to thirty percent (30%) by weight of the liquid adhesive composition. The exact amount to be used can be determined by one of ordinary skill in the art using known techniques without undue experimentation.

A preservative for use in the adhesive formulation may be paraben such as alkyl parabens and salts thereof, ethylparaben, methylparaben, methylparaben sodium, propylparaben sodium, propylparaben, butylparaben, and the like. Other suitable preservatives include hydroquinone, pyrocatechol, resorcinol, 4-n-hexyl resorcinol, benzoic acid, benzyl alcohol, chlorobutanol, dehydroacetic acid, o-phenylphenol, phenol, phenylethyl alcohol, potassium benzoate, potassium sorbate, sodium benzoate, sodium dehydroacetate, sodium propionate, sorbic acid, thimerosal, thymol, cresols, phenylmercuric compounds such as phenylmercuric borate, and phenylmercuric nitrate.

Reinforcing agents such as polymers, elastomers, fillers and fibers have been known in the art to enhance sheer, peel and tensile strength in cyanoacrylate adhesives. U.S. Pat. No. 8,287,687, the content of which is hereby incorporated by reference, summarizes this reinforcing agent art and discloses as much as a fourfold strength increase with microfiber additives in cyanoacrylates. Such reinforcing agent additives cannot flow practically through porous plugs or filters of the prior art. Examples of other suitable fibrous reinforcement include PGA microfibrils, collagen microfibrils, cellulosic microfibrils, and olefinic microfibrils.

The medical adhesive formulation may further contain small amounts of colorants such as dyes or pigments. Suitable dyes include derivatives of anthracene and other complex structures, specifically, without limitation, 1-hydroxy-4-[4-methylphenylamino]-9,10 anthracenedione (D&C violet No. 2); 9-(ocarboxyphenyl)-6-hydroxy-2,4,5,7-tetraiodo-3H-xanthen-3-one-, disodium salt, monohydrate (FD&C Red No. 3); disodium salt of 6-hydroxy-5-[(4-sulfophenyl)axo]-2-naphthalene-sulfonic acid (FD&C Yellow No. 6,); 2-(1,3dihydro-3-oxo-5-sulfo-2H-indole-2-ylidine)-2,3-dihydro-3ox- o-1H-ind-ole-5 sulfonic acid disodium salt (FD&C Blue No. 2); and 1,4-bis(4-methylanilino)anthracene-9,10-dione (D&C Green No. 6). The preferred dyes are D&C Violet No. 2, FD&C Blue No. 2, and D&C Green No. 6.

To prevent bacterial infection, the medical adhesive formulation may include an effective amount of an anti-microbial agent. The objective is to release the antimicrobial agent from the polymer film of the adhesives formed on human or animal skins to inhibit microbial growth and prevent wound or surgical site infections. Suitable antimicrobial agents include, but are not limited to, antibacterial agents such as chlorhexidine and its salts, typical antibiotics, copolymers of vinylpyrrolidone and vinyl acetate, antiseptics, iodine containing polymer such as povidone iodine, biguanidine compounds, phenol compounds such as 5-chloro-2-(2,4-dichlorophenoxy)phenol, acridine compounds, quaternary ammonium compounds such as benzalkonium chloride, cetylpridospores and zephiran, copolymers of vinylpyrrolidone and vinyl acetate cross-linked with polyisocyanates, heavy metal salts such as silver nitrate, and aldehyde compounds such as glutaraldhyde.

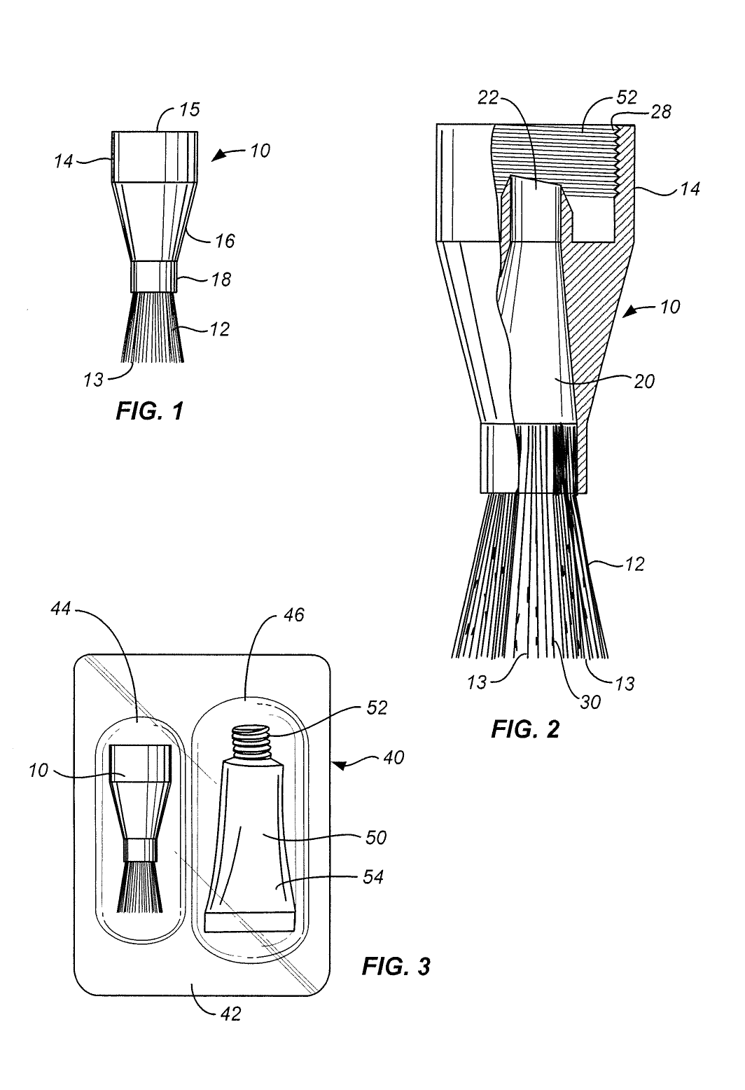

Turning now to the dispenser for storing and applying the medical adhesive formulations of the present invention, FIG. 1 illustrates a preferred brush applicator tip 10 for use in the present invention. The brush applicator tip 10 has a housing 15 and a brush 12. The housing 15 is preferably formed from a hard, molded plastic such as thermoforming polymers, polypropylene and polyethylene. These plastics may be halogenated or otherwise treated to aid their compatibility with the cyanoacrylate adhesives. In the FIG. 1 embodiment, the housing 15 has three sections, a tube interface section 14, a tapered middle section 16 and a brush holding section 18. In this embodiment, the circumference of the tube interface section 14 is larger than the brush holding section 18. The tapered design allows the brush applicator tip 10 to connect to a larger.medical adhesive tube 50 (FIG. 3) than would be possible without a tapered design. The tapered design also allows the applicator to act as a funnel to focus the flow of medical cyanoacrylates before they reach the brush 12.

The brush 12 is formed from a plurality of fibers or bristles 13. The bristles 13 are preferably formed from a hard, heat fusible plastic, such as nylon, polyester, polyethylene or polypropylene and can preferably number anywhere from a dozen to well over a hundred. These plastics may be halogenated or otherwise treated to aid their compatibility with the cyanoacrylate adhesives. As shown in FIG. 1, the bristles 13 should preferably flare outwardly at their free end. The bristles 13 should be dense enough to allow the medical adhesive formulation to contact the bristles as the formulation is flowing out of the tube but not so dense as to prevent the medical cyanoacrylate formulation from flowing past the bristles. Kay's U.S. Pat. No. 4,907,841, the disclosure of which is herein incorporated by reference, describes a method of making a brush applicator tip and brush bristles in a way that could be used in the present invention. By using the applicator brushes of the present invention, one can work with higher viscosity medical adhesives and thixotropes, such as fumed silica, that would not be possible with the porous filter plugs of the prior art.

FIG. 2 is a close-up, cutaway view of the brush applicator tip 10. In this embodiment, the tube interface section 14 preferably has internal threads 28 which are sized to mate with the external threads 52 of the medical adhesive tube 50. An internal cone 20 may also be included which has a cutting tip 22 that can be used to break a seal formed on the cyanoacrylate tube 50.

The brush bristles 12 are preferably coated or impregnated with a dry, solvent-free initiator and/or accelerator 30 to facilitate curing of the cyanoacrylate adhesives. A cyanoacrylate initiator helps begin the curing of the cyanoacrylate through polymerization or cross-linking. By comparison, a cyanoacrylate accelerator increases the rate of cyanoacrylate curing through polymerization or cross-linking. Suitable polymerization initiators and/or accelerators 30 may include, but are not limited to, calixarenes and oxacalixarenes, silacrowns, crown ethers, cyclodextrin and its derivatives, polyethers, aliphatic alcohol, various aliphatic carboxylic acid esters, benzoyl peroxide, amine compounds such as, but not limited to, triethyl amine, diethyl amine, butyl amine, isopropyl amine, tributyl amine, N,N,-dimethyl aniline, N,N-diethyl aniline, N,N-dimethyl-p-toluidine, N,N-dimethyl-m-toluidine, N,Ndimethyl-o-toluidine, dimethyl benzyl amine, pyridine, picoline, vinyl pyridine, ethanolamine, propanolamine and ethylene diamine, phase transfer catalysts, quaternary ammonium salts such as alkyl ammonium salts, amide-bonded ammonium salts, ester-bonded ammonium salts, ether-bonded ammonium salts and alkylimidazolinium salts, cyclosulfur compounds and derivatives, and polyalkylene oxides and derivatives. Prior to use, the initiator and/or accelerator 30 should be solvent-free to avoid having the solvent slow down polymerization of the medical adhesives after the medical adhesives contact the brush bristles.

The initiator and/or accelerator may be applied to the brush bristles during manufacture by spraying, dipping or immersion of the brush bristles within a liquid medium containing the initiator and/or accelerator. During the manufacturing process, the liquid medium may include solvents, such as ether, acetone, ethanol, pentane or mixtures thereof. To the extent solvents are used during the manufacturing process, they need to be evaporated before the initiator/accelerator is ready for use in order to avoid having the solvent slow down polymerization of the medical adhesives.

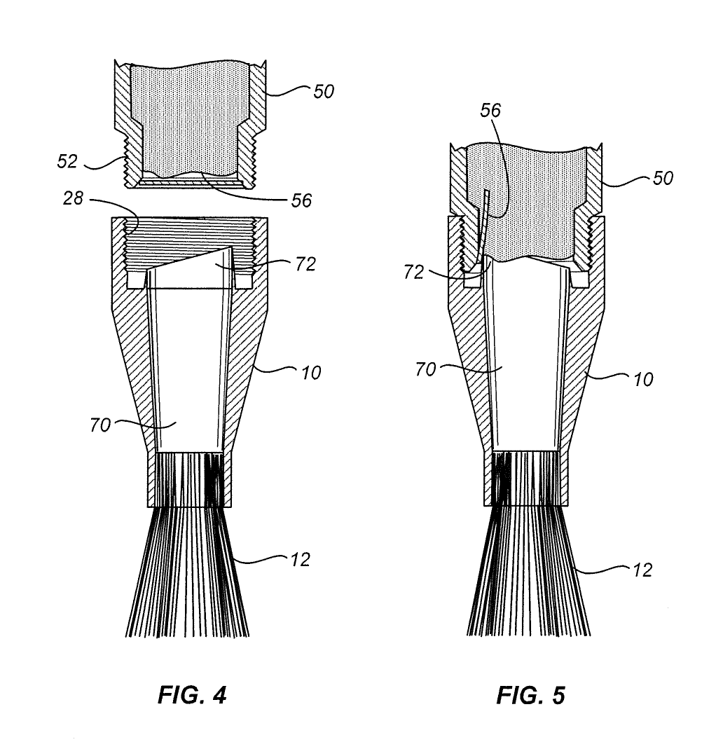

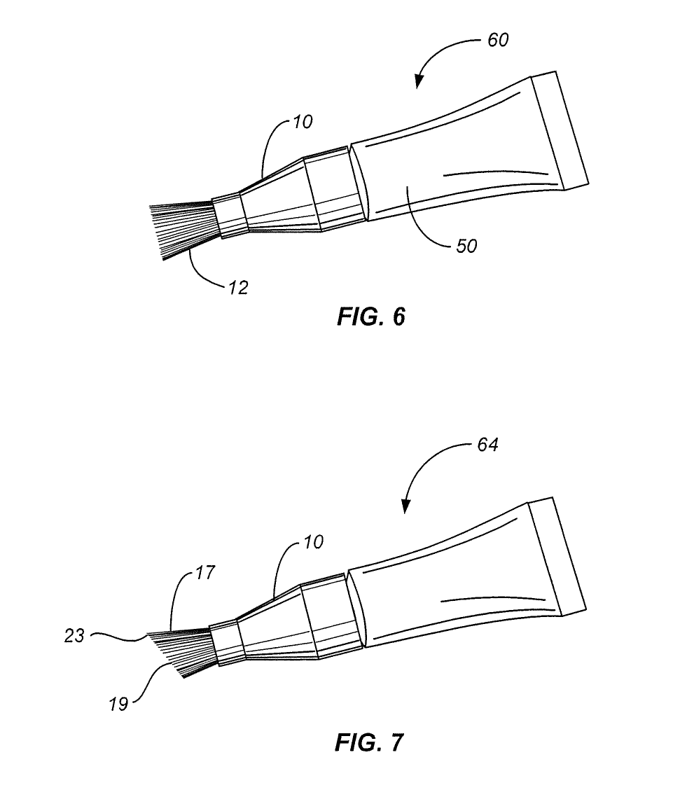

FIG. 3 shows an unassembled dispenser (see, FIG. 4) of the present invention packaged in a kit form 40. The dispenser consists of brush applicator tip 10 and a container, in this case a medical adhesive tube 50. Suitable materials for the tube 50 include aluminum, plastic, laminated aluminum foil and multi-layer sheet materials. Suitable plastics may include, but are not limited to, high density polyethylene (HDPE), polypropylene, polyvinylchloride, acrylonitrile copolymer, polycarbonate, polytetrafluoroethylene (PFTE), polyethylene terephthalate (PET), polystyrene (PS), and polymethylpentene. Multi-layer sheet materials are typically composed of at least two layers of such metal or plastic materials. In the FIG. 3 embodiment, the tube 50 is formed from aluminum and has a container section 54, which holds the medical adhesive, an external threaded section 52 and a seal 56 (see, FIG. 5).

The aluminum tube 50 embodiment shown in FIG. 3 is particularly preferred. It is well known in the cyanoacrylate monomer art that aluminum packaging provides the longest shelf life of any cyanoacrylate packaging material. This is especially so during sterilization procedures where greater viscosity stability is observed for aluminum packaging than for the frangible glass of prior art systems.

In the FIG. 3 embodiment, the brush applicator tip 10 and medical adhesive tube 50 are separated into separate compartments 44, 46 which are preferably formed from thin, see through plastic. Keeping the brush applicator tip 10 and medical adhesive tube 50 separate prior to use has several advantages. Through separation, the medical adhesive tube 60 can remain in a sealed, sterilized condition for a substantial period of time. This allows the medical adhesive to have a longer shelf life. Sealing of the medical adhesive tube 50 also prevents the medical adhesive from prematurely coming in contact with the initiator/accelerator coating 30 on the brush bristles. To save expense, the kit 40 preferably uses a sterile cardboard-like backing 42 for the see through plastic compartments 44, 46 to hold the brush applicator tip 10 and medical adhesive tube 50 in place.

FIGS. 4 and 5 illustrate how the medical adhesive tube 50 and brush applicator tip 10 of the present invention can be joined together to allow the medical adhesive to flow through the brush bristles 12 and applied to heal a wound. As shown in FIG. 4, the medical adhesive tube 50 and brush applicator tip 10 should first be axially aligned so that external threads 52 of the medical adhesive tube 50 can be inserted into the cylindrical space created by the internal threads 28 of the brush applicator tip 10. When the threads 28, 52 make contact and the medical adhesive tube 50 is screwed into the brush applicator tip 10 as shown in FIG. 5, a hollow internal tube 70 with a cutting edge 72 is preferably provided in the brush applicator tip to simultaneously cut away the medical adhesive tube seal 56. Once the seal 56 is cut away, the medical adhesive can flow from the medical adhesive tube 50 into the brush applicator tip 10 through the hollow internal tube 70 of the brush applicator tip 10. Once the medical adhesive flows through the hollow internal tube 70, it comes in contact with the brush bristles 12 of the brush applicator tip 10 where it can be applied to heal a wound.

FIG. 6 shows an assembled dispenser 60 of the present invention which is constructed by fully screwing the threads of the medical adhesive tube 50 into the threads of the brush applicator tip 10. FIG. 7 shows an alternative embodiment for the fully assembled dispenser 64 where, instead of being flat, the free end 19 of the applicator tip bristles are tapered. Through use of such tapering, a point 23 is created on the brush which can be used to apply the medical adhesives of the present invention with great precision.

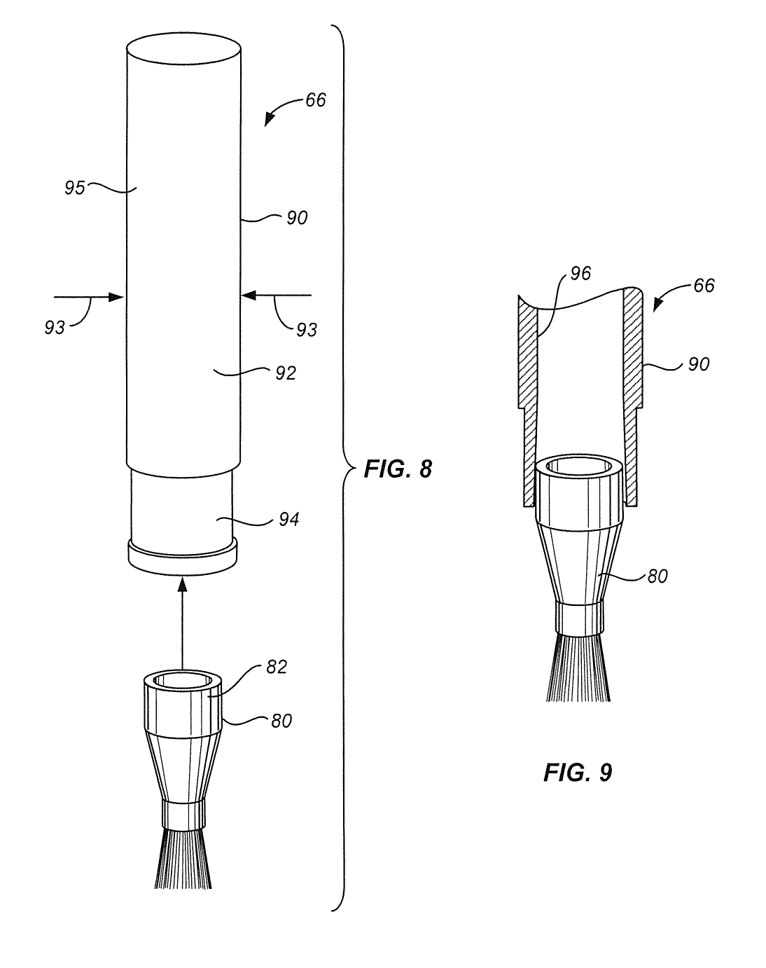

FIGS. 8 and 9 show a further alternative embodiment for the dispenser 66 of the present invention. In these figures, a plastic tube 90, rather than an aluminum tube, is used to hold the medical adhesives. In this embodiment, the plastic tube 90 has two concentric sections 92, 94 with the lower section 94 being movably inserted into the upper section 92. These concentric sections 92, 94 allow medical adhesives to be squeezed out of the plastic tube 90 by moving the lower section 94 upward into the upper section 92. Medical adhesives may also be squeezed out of the plastic tube 90 by applying circumferential pressure 93 on the exterior walls 95 of the plastic tube 90.

Before medical adhesive is squeezed out of the plastic tube 90, the plastic tube 90 should be assembled with the brush applicator tip 80 as shown in FIG. 9. As compared with the FIGS. 4 and 5 embodiments, there are no threads on the plastic tube 90 and brush applicator tip 80 in this embodiment to screw together. Instead, a friction fit is made between the exterior surface of the tube interface section 82 of the brush applicator tip 80 and the internal surface 96 of the plastic tube's lower section 94. Preferably, as this friction fit is made, a seal (not shown) is simultaneously broken on the plastic tube's lower section to allow medical adhesive to flow out of the plastic tube 90 and into the brush applicator tip 80.

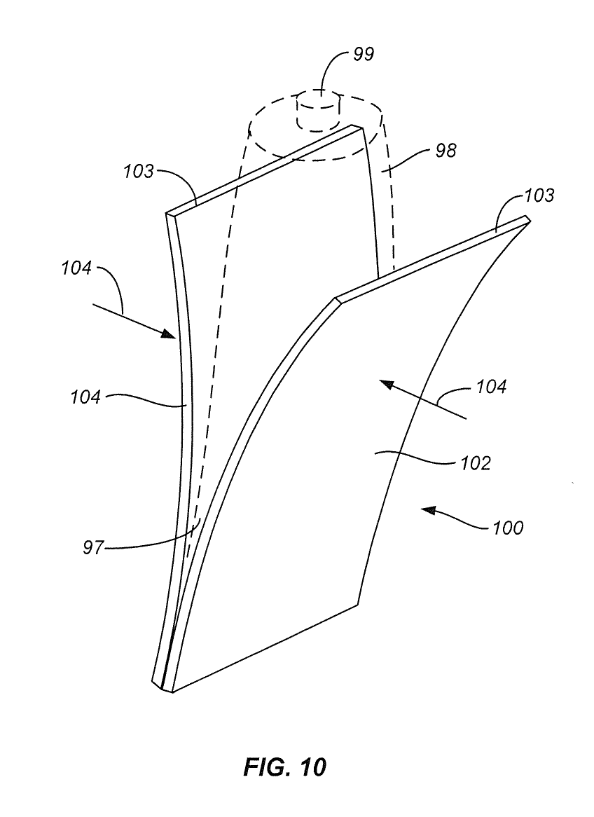

FIG. 10 illustrates a bellows 100 accessory which can be advantageously used to allow medical adhesives to flow more evenly out of the tube container 98 holding the medical adhesive. In this embodiment, the bellows consists of two curved sheets 102 which are joined at one end so that the free ends 103 of each sheet flare away from each other. The sheets 102 are preferably formed of a hard, flexible material such as metal or a hard plastic. In operation, the medical adhesive tube container 98 is placed between the two sheets 102 with the closed end 97 of the tube container being held between where the sheets join and the dispensing end 99 of the tube container 98 facing away from where the sheets join. After the dispensing end 99 of the tube container 98 is connected to a brush applicator tip (not shown), the bellows sheets can be pressed together 104 to more evenly dispense the medical adhesive.

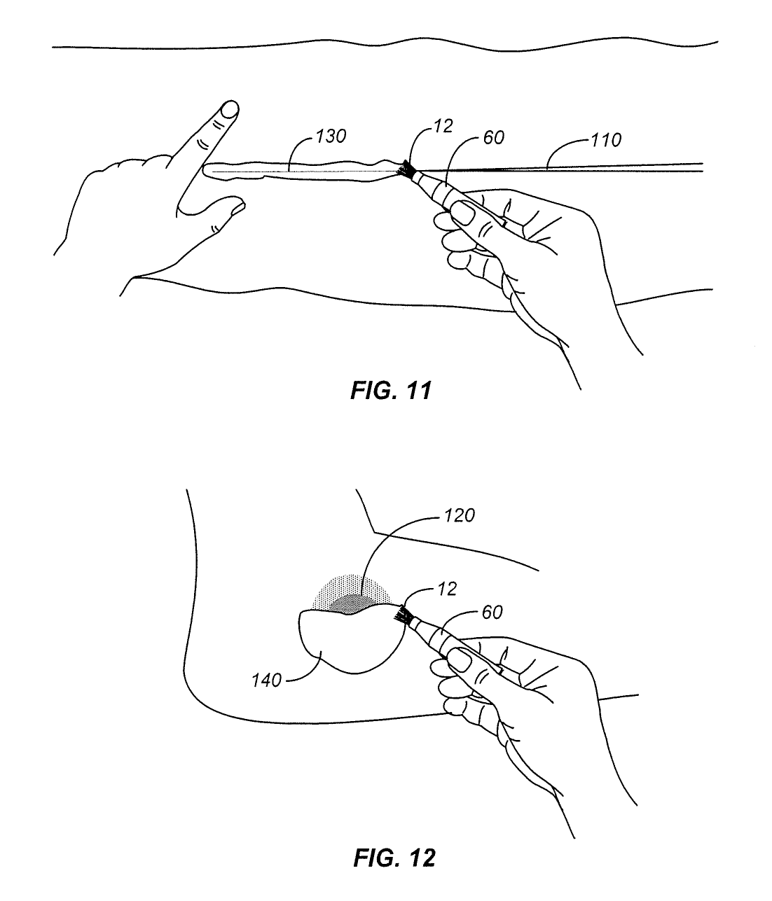

FIGS. 11 and 12 illustrate how the medical adhesive compositions of the present invention can be used to heal wounds using the dispensers of the present invention. In FIG. 10, the wound is a long cut 110. In the prior art DERMABOND® system, contact of the porous plug applicator with the wound is to be avoided because such contact could further damage the wound. By comparison, in the present system, the brush 12 may gently contact the wound 110 as medical adhesive 130 is being applied to allow for more a precise application of the adhesive. In the case of the cut wound 110 shown in FIG. 10, the skin should be pressed together (i.e., abutted) to close the cut wound before the medical adhesive is then applied on top of the closed cut wound. When the brush 12 gently contacts the wound in the present invention, it can spread medical adhesive over a wider surface area than is possible for the DERMABOND® porous plug. This means that medical adhesive needs to be applied over the wound fewer times with the brush 12 of the present invention than is needed if the DERMABOND® porous plug is used.

A further advantage of the present invention is that the medical adhesive can be formulated with a high enough viscosity to allow for a controlled application. In the DERMABOND® system, the viscosity needs to be low enough to allow the medical adhesive to pass through the porous plug applicator tip. With lower viscosities, one can get messy, uncontrolled runniness of the medical adhesive.

FIG. 12 shows the medical adhesive compositions of the present invention being used to create a seal 140 over an abrasion wound 120, bedside ulcer or other type of wound that covers a larger area. Again, as medical adhesive is squeezed out of the dispenser 60, the dispenser bristles 12 can gently touch the wound to apply the adhesive in a controlled manner. With higher viscosity adhesives, the seal created over the larger area wound typically has more tensile strength than the seal created by a lower viscosity adhesive. In other words, it becomes an inexpensive liquid bandage. Periodic repair of the seal by rebrushing can produce longevity up to 28 days for the seal and maintain a functioning, open space wound drain if required.

In addition to the medical applications illustrated in FIGS. 11 and 12, the medical adhesives and dispensers of the present invention may be used for closing surgical incisions, dressing traumatically lacerated tissues, dressing bums, covering superficial or skin surface wounds and applying onto surgical incision sites before surgery to provide in situ and post-surgery inhabitation of surgical site infections. They may also be used for binding other materials such as plastics, metals, wood, ceramics, fabrics, paper and the like.

If desired, the medical adhesives of the present invention can be formulated to be bio absorbable. The term “bio absorbable” refers to polymers or medical devices that are able to be completely degraded, eroded and/or gradually absorbed or eliminated by the body when such polymers or medical devices are exposed to body fluid such as blood. Bio absorbable adhesives can be used in many different applications including, but not limited to, general wound closure, endoscopic surgery, cardiac surgery, hernia surgery, arthroscopic surgery. Bio absorbable adhesives are preferably based on cyanoacrylates. Efforts to increase the tissue compatibility of alpha-cyanoacrylates have included modifying the alkyl ester group of the cyanoacrylates. For example, increasing the alkyl ester chain link to form the higher cyanoacrylate analogs, e.g., butyl-2-cyanoacrylates and octyl-2-cyanoacrylates, has been found to improve biocompatibility but the higher analogs biodegrade at slower rates than the lower alkyl cyanoacrylates. A bio absorbable adhesive composition can be composed of alkoxyalkyl cyanoacrylate and polyethylene glycol. These bio absorbable adhesive compositions can also consist of the mixture of alkyl cyanoacrylate, alkoxyalkyl cyanoacrylate and polyethylene glycol. A preferred alkoxyalkyl cyanoacrylate is methoxyisopropyl cyanoacrylate. Other bio absorbable adhesives may include copolymers of alkyl cyanoacrylate or alkoxyalkyl cyanoacrylate with other biocompatible monomers such as trimethylene carbonate, alkylene glycol, glycolide, lactide, epsilon, caprolactone and dioxane. Dispersed proteins also aid bio absorbability.

For medical applications, it is important that the medical adhesives and dispensers of the present invention be sterilized prior to use. Heat or irradiation sterilization techniques, such as electron beam sterilization, gamma sterilization, and/or X-ray sterilization, may be used. Aluminum tubes for the medical adhesives are preferred because they can be more easily sterilized than other types of containers, such as the DERMABOND® frangible glass vials. Squeezable plastic tubes are also suitable because they remain stable during heat and/or irradiation sterilization. Coextruded or laminated polymer films are additionally suitable. Reference is made to films laminated with acrylonitrile copolymer as the inner layer to contact cyanoacrylate adhesives in U.S. Pat. No. 8,550,737. Such materials are irradiation stable under the maximum dosage of e-beam, gamma and X-ray sterilization. The dose of heat or irradiation applied should be sufficient to sterilize both the impregnated brush applicator and the adhesive tube.

Another way of sterilizing the medical adhesives before they are inserted into a dispenser tube is to filter them through, for example, a 0.2 μm filter. The impregnated brush applicator and adhesive tube may also be sterilized with heat and/or ethylene oxide. A sterility assurance level (SAL) should be obtained at a minimum of 10⁻³, which means that the probability of a single unit being non-sterile after sterilization is 1 in 1000. In more preferred embodiments, the sterility assurance level should be at least 10⁻⁶. The sterility of the cyanoacrylate adhesives packaged in various containers after irradiation sterilization may be analyzed by bacteriostasis and fungistasis tests. After testing with challenging microorganisms such as Bacillus subtilis, Candida albicans and Aspergillus niger, no growth of the microorganisms for the cyanoacrylate adhesive in the impregnated brush applicator and adhesive tube after irradiation sterilization indicates the sterility of the cyanoacrylate adhesive.

Sterilization of the medical adhesive when it is in an aluminum or plastic tube by heat, e-beam, gamma or X-ray sterilization does not cause the medical adhesive to prematurely cure. For example, the medical adhesive in an aluminum or plastic tube after heat or irradiation sterilization may have a shelf life of at least 12 months, sometimes even 24 months. The shelf life stability of packaged medical adhesive formulations of the present invention sterilized by heat or irradiation sterilization techniques may be evaluated by an accelerated aging study at 80° C. In U.S. Pat. No. 8,550,737, a study was performed in an oven at 80° C. for a period of 13 days. Based on ASTM F 19802, 13 days accelerated aging at 80° C. correlates to 2 years of shelf life at ambient temperatures and 1 day of accelerated aging at 80° C. is equal to 56 days of shelf life at ambient temperature.

### EXAMPLES

### Example 1

### Toughness of Thickened Medical Adhesive Formulations

When the medical adhesives of the present invention are used to seal a wound, it is important that they make a tough seal which can be elongated without breaking. To create optimum medical adhesive formulations, a 2-octyl cyanoacrylate monomer was mixed with various thickeners in different concentrations. As a control, one medical adhesive formulation was made without a thickener. Two formulations were then made with polyvinyl acetate (PVAc) thickeners at concentrations of 5% and 8%. Two formulations were also made with poly 2-ethyl hexyl methylmethacrylate (PEHMA) thickeners at concentrations of 5% and 8%. After casting and curing, the medical adhesive formulations were then tested for viscosity and percent elongation before breaking. The test results are presented below in Table 1. Medical adhesive formulations with higher viscosity and higher elongation strength provide a tougher seal. For comparison purposes, DERMABOND®'s regular medical adhesive product has a viscosity of 50 cps and DERMABOND®'s “high viscosity” product has viscosity of 100 cps. By using a brush applicator of the present invention, rather than DERMABOND®'s porous plug, the medical adhesives of the present invention can be made with a higher viscosity to produce greater toughness.

TABLE 1
Toughness

| Monomer | Thickener | % Elongation | Viscosity (cps) |
|---|---|---|---|
| 2-octyl CA#2 | None | 222 | 3 |
| 2-octyl CA#2 | +PVAc 5% | 367 | 79 |
| 2-octyl CA#2 | +PVAc 8% | 850 | 221 |
| 2-octyl CA#2 | +PEHMA 5% | 278 | 76 |
| 2-octyl CA#2 | +PEHMA 8% | 378 | 132 |

In addition to toughness, another benefit of the higher viscosity medical adhesive formulations of the present invention is the increased ability to control the moisture vapor transmission rate of the cured materials. U.S. Patent Application Pub. No. 2011/0117047 discusses the importance of moisture vapor transmission rate (MVTR) of cyanoacrylate medical adhesive compositions. While cyanoacrylate compositions have been previously known for surgical wound dressing and management, there is a need to exhibit desirable permeability or breathability for effective healing. Permeability is a desirable characteristic for medical dressings because it prevents maceration of the skin due to trapped moisture, improves wound healing and enhances the patient's comfort while the bandage is being worn. Permeability can provide further benefits such as: (a) removing and preventing exudates from pooling while keeping the wound moist during the process of healing, (b) permitting appropriate oxygen ingress and carbon dioxide egress, and (c) minimizing the formation of trauma to the surrounding or new tissue. With high MVTR, the wound dressing possesses improved fluid handling capacity and prevents wound desiccation (see, U.S. Pat. No. 6,495,229 and Hansen et al. Adhesives Age, 22-25, 2003). Specially engineered fibers and inorganic fillers advantageously change the morphology of the cyanoacrylates formulations to create micro porous films. These thickening additives cannot be used practically with porous plugs of the DERMABOND® product.

### Example 2

### Flow Control Through Higher Viscosities

To effectively apply medical adhesives to a wound, it is important that the medical adhesives be viscous enough that they will not run uncontrollably. The runniness of five medical adhesive formulations of increasing viscosity were tested. For the test, 0.5 ml. of each medical adhesive was placed on a glass plate inclined on a 45° plane. The time was then measured for the medical adhesive to flow 40 millimeters. The results of the flow control test are presented below in Table 2. “DBHV” is DERMABOND's “high viscosity” medical adhesive product. “8% PVA in Octyl # 2” is a 2-octyl cyanoacrylate monomer formulation with a polyvinyl acetate (PVAc) thickener added at a concentration of 8%. “CB X” and “CB Y” are commercial cyanoacrylate products produced by Cyberbond LLC. “CB Gel” is a cyanoacrylate thixotropic gel product produced by Cyberbond LLC. As shown in Table 2, the higher viscosity medical adhesive formulations are less runny.

TABLE 2
Runniness

| | | Flow in millimeters |
|---|---|---|
| | Viscosity | over Time in seconds |
| DBHV | 90 cps. | 40 mm in 8 sec. |
| 8% PVA in | 221 cps. | 40 mm in 86 sec. |
| Octyl# 2 | | |
| CB X | 1600 cps. | 40 mm in 111 sec. |
| CB Y | 3500 cps. | 10 mm in 238 sec. |
| CB Gel | Gel | 10 mm in 840 sec. |

In the foregoing specification, the invention has been described with reference to specific preferred embodiments and methods. It will, however, be evident to those of skill in the art that various modifications and changes may be made without departing from the broader spirit and scope of the invention as set forth in the appended claims. The specification and drawings are, accordingly, to be regarded in an illustrative, rather than restrictive sense; the invention being limited only by the appended claims.

What is claimed is:

1. A medical adhesive dispenser comprising:
a container storing a medical adhesive formulation;
an applicator having brush bristles coated and/or impregnated with a dry, solvent-free initiator and/or accelerator capable of curing said medical adhesive formulation;
wherein said container and applicator, when connected, are configured to allow said medical adhesive formulation to pass from said container, through said applicator and onto said applicator brush bristles.

2. The medical adhesive dispenser of claim 1 wherein said medical adhesive formulation includes a cyanoacrylate monomer.

3. The medical adhesive dispenser of claim 2 wherein said cyanoacrylate monomer includes 2-octyl cyanoacrylate monomer and/or n-butyl-2-cyanoacrylate monomer.

4. The medical adhesive dispenser of claim 1 wherein said medical adhesive formulation includes a thickener.

5. The medical adhesive dispenser of claim 4 wherein said thickener is polyvinyl acetate (PVAc) or poly 2-ethyl hexyl methylmethacrylate (PEHMA)

6. The medical adhesive dispenser of claim 1 wherein said medical adhesive formulation includes one or more of the following additives: stabilizers, plasticizing agents, preservatives, reinforcing agents, colorants and anti-bacterial compounds.

7. The medical adhesive dispenser of claim 1 wherein said container is made from aluminum, plastic, laminated aluminum foil or multi-layer sheet materials.

8. The medical adhesive dispenser of claim 2 wherein said initiator and/or accelerator is selected from the group consisting of calixarenes and oxacalixarenes, silacrowns, crownethers, cyclodextrin, polyethers, aliphatic alcohol, various aliphatic carboxylic acid esters, benzoyl peroxide, triethyl amine, diethyl amine, butyl amine, isopropyl amine, tributyl amine, N,N,-dimethyl aniline, N,N-diethyl aniline, N,N-dimethyl-p-toluidine, N,N-dimethyl-m-toluidine, N,Ndimethyl-o-toluidine, dimethyl benzyl amine, pyridine, picoline, vinyl pyridine, ethanolamine, propanolamine and ethylene diamine, phase transfer catalysts, alkyl ammonium salts, amide-bonded ammonium salts, ester-bonded ammonium salts, ether-bonded ammonium salts and alkylimidazolinium salts, cyclosulfur compounds and polyalkylene oxides.

9. The medical adhesive dispenser of claim 1 wherein said container can be attached to said applicator by a screw connection.

10. The medical adhesive dispenser of claim 9 wherein said container further includes a seal which is broken by said applicator when said tube and applicator are screwed together.

11. The medical adhesive dispenser of claim 1 wherein said medical adhesive formulation is bio-absorbable and/or bio-degradable.

12. A medical adhesive dispenser comprising:
a container having a medical adhesive formulation including a cyanoacrylate monomer and a thickener;
an applicator having exposed brush bristles coated and/or impregnated with a dry, solvent-free initiator and/or accelerator capable of curing said cyanoacrylate;
wherein said tube and applicator, when connected, are configured to allow said medical adhesive formulation to pass out of said container, through said applicator and onto said applicator brush bristles.

13. The medical adhesive dispenser of claim 12 wherein said cyanoacrylate monomer includes 2-octyl cyanoacrylate monomer and/or n-butyl-2-cyanoacrylate monomer.

14. The medical adhesive dispenser of claim 12 wherein said thickener is selected from the group consisting of poly 2-ethyl hexyl methylmethacrylate (PEHMA), polycaprolactone, polyvinyl acetate (PVAc), copolymers of alkylacrylate and vinyl acetate, polyalkyl methacrylates, polyalkyl acrylates, lactic-glycolic acid copolymers, lactic acid-caprolactone copolymers, polyorthoesters, copolymers of alkyl methacrylates and butadiene, polyoxalates, and triblock copolymers of polyoxypropylene flanked by two hydrophilic chains of polyoxyethylene.

15. The medical adhesive dispenser of claim 12 wherein said medical adhesive formulation includes 2-octyl cyanoacrylate and up to 8% polyvinyl acetate (PVAc) or poly 2-ethyl hexyl methylmethacrylate (PEHMA) thickener.

16. The medical adhesive dispenser of claim 12 wherein said container is an aluminum tube.

17. The medical adhesive dispenser of claim 12 wherein said container has a pierceable seal and said applicator has a cutter capable of piercing said container seal when said container and applicator are joined together.

18. A medical adhesive kit comprising:
a container having a medical adhesive formulation including a cyanoacrylate monomer and a thickener;
an applicator having exposed brush bristles coated and/or impregnated with a dry, solvent-free initiator and/or accelerator capable of curing said cyanoacrylate monomer; and,
packaging capable of holding said container and applicator separately from one another.

19. A method of treating a wound comprising:
selecting a medical adhesive dispenser with a stored medical adhesive formulation and a brush applicator wherein the bristles of said brush are coated and/or impregnated with a dry, solvent-free initiator and/or accelerator;
dispensing said stored medical adhesive formulation onto said brush bristles;
applying said medical adhesive formulation onto said wound by gently brushing said medical adhesive coated brush bristles onto said wound.

20. The method of claim 19 wherein, when said wound is a cut, the cut is abutted before medical adhesive is applied over said cut.

21. The method of claim 19 wherein, when said wound is an abrasion or ulcer, the medical adhesive formulation is applied directly over said abrasion or ulcer to form a seal.

22. The method of claim 19 wherein said medical adhesive formulation includes a cyanoacrylate monomer.

23. The method of claim 22 wherein said cyanoacrylate monomer includes 2-octyl cyanoacrylate monomer and/or n-butyl-2-cyanoacrylate monomer.

24. The method of claim 22 wherein said medical adhesive formulation is bio-absorbable and/or bio-degradable.

25. The method of claim 19 wherein said medical adhesive formulation includes one or more of the following additives: stabilizers, plasticizing agents, preservatives, reinforcing agents, colorants and anti-bacterial compounds.
